(19) United States
(12) Patent Application Publication
(10) Pub. No.: US 2025/0032653 A1
(43) Pub. Date: Jan. 30, 2025

(54) Electrically-Activated Anti-Pathogen Membrane

(71) Applicants: Michael G. McAlister, Walnut Creek, CA (US); Gerard McDermott, Orinda, CA (US); Kristofer Pister, Orinda, CA (US)

(72) Inventors: Michael G. McAlister, Walnut Creek, CA (US); Gerard McDermott, Orinda, CA (US); Kristofer Pister, Orinda, CA (US)

(21) Appl. No.: 18/761,243

(22) Filed: Jul. 1, 2024

Related U.S. Application Data
(60) Provisional application No. 63/524,371, filed on Jun. 30, 2023.

(51) Int. Cl. A61L 2/03; A61L 2/24; A61L 2/26

(52) U.S. Cl. CPC A61L 2/03; A61L 2/24; A61L 2/26; A61L 2202/11; A61L 2202/14

(57) ABSTRACT

A membrane system comprising an arrangement of conductors, electrically attachable to a power supply, encapsulated within a protective material, which conductors, when powered, may create an electrical field that is hostile to various types of pathogens and pathogen vectors, such as microbial, viral, bacterial, biologic, animal, and insect.

### CROSS-REFERENCE TO RELATED APPLICATIONS

This application claims the benefit of U.S. Application No. 63/524,371, filed on Jun. 30, 2023, by the present inventors, entitled “Electrically-Activated Anti-Microbial Membrane,” which is hereby incorporated by reference in its entirety for all allowable purposes, including the incorporation and preservation of any and all rights to patentable subject matter, such as features, elements, processes and process steps, improvements, and their descriptions that may supplement or relate to the subject matter described herein.

### RESEARCH OR DEVELOPMENT

Not Applicable

### BACKGROUND OF THE INVENTION

This innovation relates generally to methods or apparatus for neutralizing contaminants on or sterilizing a surface, and more specifically to an arrangement of electrodes disposed within an encapsulating material, which when energized create a localized electrical field affecting the area at and above the surface of the material.

Society has become more aware of the risks posed by germs. As such, society continues to seek effective ways of preventing the spread of microbes responsible for infections. High-contact areas are seen as a significant vector for the spread of microbes, and therefore infection. Some research in the general field includes articles by various experts in the field, such as: “Antimicrobial Treated Devices”, by Leviton, Inc., 2020, found at https://www.leviton.com/en/solutions/industries/health-care/antimicrobial; “Sterilization Handle”, by Seo Seon Hyeong et al., 2014, found through Lens.org (https://www.lens.org/); “Sterilization system using microwave and UV light”, by S. Iwaguch et al., 2002, found through Elsevier Group (https://www.elsevier.com/); “Sterilization of gripping surfaces”, by Kelly L. Jones (U.S. Pat. No. 7,175,607) using germicidal light on a gripping surface; “Static electricity powered copper oxide nanowire microbicidal electroporation for water disinfection,” by Chong Liu et al., found in Nano Letters 2014, 14, 10, 5603-5608; “Comparison of Sterilization efficiency of pulsed and continuous UV light using tunable frequency UV system,” by W. Luo et al., 2014, found through Elsevier Group (https://www.elsevier.com/); and “Efficient Sterilization of bacteria by pulse electric field in micro-gap”, by Satoshi Uchida, Makoto Houjo, and Fumiyoshi Tochikubo, Journal of Electrostatics 66.7-8 (2008): 427-431.

It would be an improvement to the field of art to have a membrane affixable to a surface that could create an environment hostile to microbial, viral, bacterial, and small animal or insect contamination. It would be an improvement to the field of art to have a membrane affixable to a surface that could either or both reduce a microbial load and inhibit biofilm formation on a surface. It would further be an improvement to be able to integrate such membrane material into the surface of a high-contact area.

### BRIEF DESCRIPTION OF THE DRAWINGS

FIG. 1 is a schematic view of an exemplary membrane system with a segment of a membrane material, according to the present invention, not shown to scale.

FIG. 2 is a schematic, cross-sectional side view of the material segment of FIG. 1, cut at line A-A.

FIG. 3 is a side-view illustration of an exemplary embodiment, according to the present invention, disposed on the contact surface of a door handle.

### DESCRIPTION OF THE PREFERRED EMBODIMENT

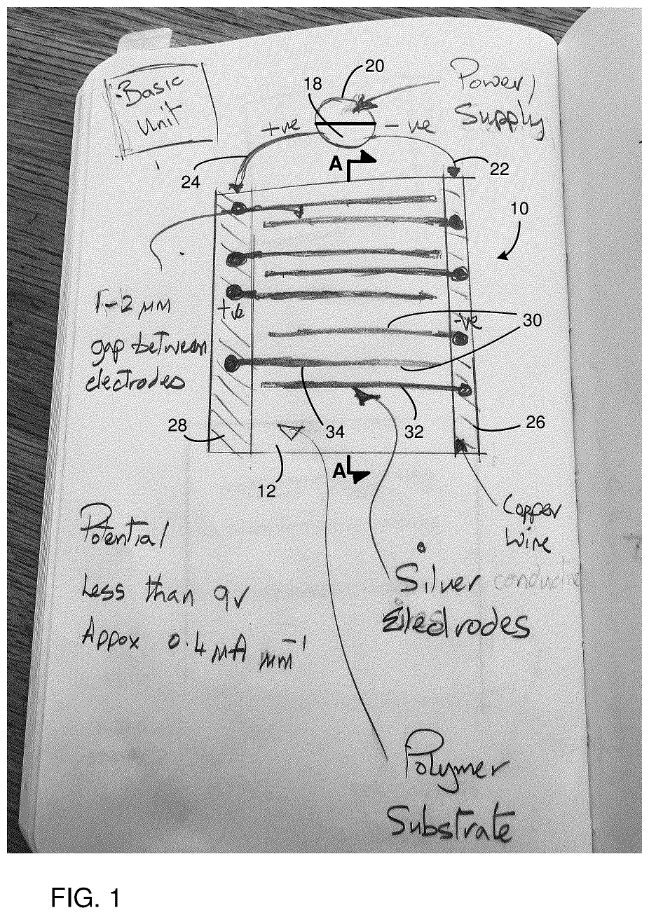

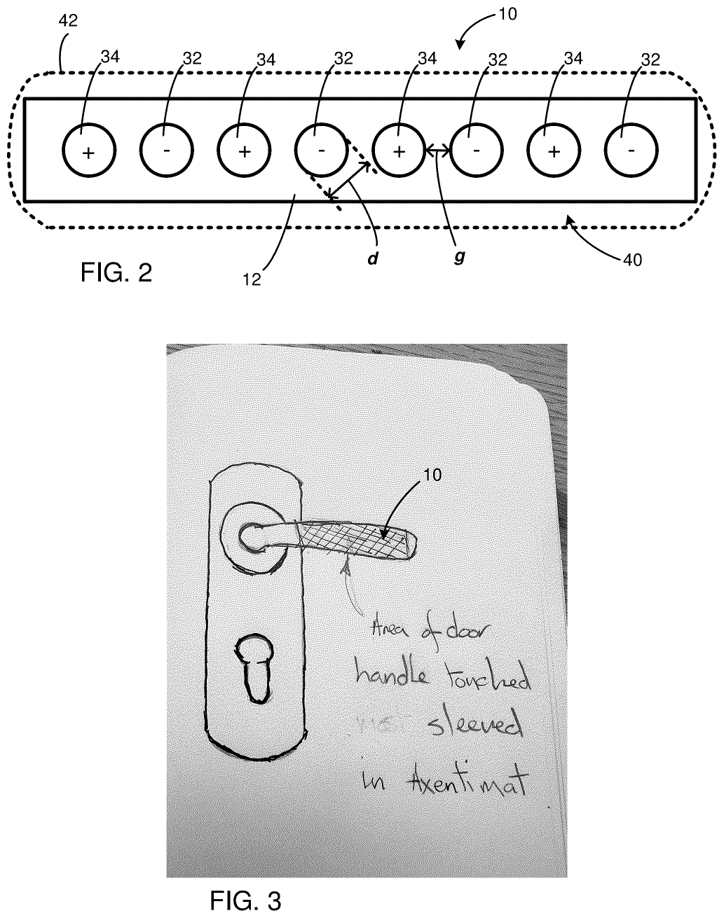

Referring now primarily to FIGS. 1 and 2, the present exemplary membrane 10 may be embodied in a structure that comprises an arrangement of electrodes 30, potentially referred to in this disclosure as an array, encased in a protective or encapsulating compound or material 12. In an exemplary embodiment, the electrodes 30 may be arranged as a layer of interdigitated conductors. The membrane 10 may create an electrical field hostile or destructive to various types of pathogens or pathogen vectors, including without limitation microbial, viral, bacterial, biologic, animal, and insect. The created electrical field may reduce a microbial load and inhibit biofilm formation on a surface. A portion of the electrical field, which effectively neutralizes or repels contaminants to a level where they may have little to no deleterious effect on humans, may be referred to as an efficacious area or efficacious zone 40. The efficacious area or efficacious zone 40 may be defined as the region within a boundary 42 to which effective protection extends. Because of this, contaminants and pathogen threats to human health via contact with contaminated surfaces and environments may be significantly reduced within the efficacious zone 40. In an exemplary embodiment, other arrangements of electrodes 30 within a membrane 10 may include a micro-mesh of interlaced electrodes 30. Other arrangements of electrodes 30 may be suitable to create an efficacious zone 40 within the scope of this disclosure and subsequently allowed claims.

Examples of contaminants on which the membrane 10 may be effective may include viruses, which may have general diameter sizes of around 100 nanometers, prokaryotic pathogens, possibly grouped as bacteria, which may have general diameter sizes of around 500 nm, and eukaryotes, possibly grouped as fungi, which may have general diameter sizes of around 10 to 15 microns. The membrane 10 may also be effective on other types and sizes of contaminants. An electrical field of the membrane 10 may establish an efficacious zone 40 that extends to the surface of the membrane 10 and beyond to encompass the size of the target organisms and contaminants.

The mode of action within the efficacious zone 40 may be to disrupt the function, reproduction, and ability to affect or infect humans. The mode of action within the efficacious zone 40 may be to prevent the formation of bacterial or yeast biofilms, and thereby increase the efficacy of any of an assortment of antimicrobial agents and chemical treatments that may neutralize contaminants on a surface of the membrane 10 and the surface on which the membrane 10 is disposed or integrated.

An exemplary membrane 10 may comprise an encapsulating material 12 and a plurality of electrodes 30 positioned within the encapsulating material 12. In an exemplary embodiment, the membrane 10 may be electrically connected to a power supply or source 20, which may be either integrated into the construction of the membrane 10 or independent of the membrane 10. In an exemplary embodiment, the power supply 20 may be electrically connected to the plurality of electrodes 30 by a negative feed line 22 and a positive feed line 24. In an exemplary embodiment, the negative feed line 22 may be electrically connected to a negative lead 26 and the positive feed line 24 may be electrically connected to a positive lead 28. In an exemplary embodiment, the electrical connection between the power supply 20 and the plurality of electrodes 30 may include the connection of the power supply 20 to a negative feed line 22 and a positive feed line 24, which are connected to a negative lead 26 and a positive lead 28, respectively, which are each connected to at least one unique electrode 30 of the plurality of electrodes 30. In an exemplary embodiment, an electrode 30 that is electrically connected to the negative feed line 22 may be considered a negative electrode 32, and an electrode 30 that is electrically connected to the positive feed line 24 may be considered a positive electrode 34. In an exemplary embodiment, an electrode 30 may have a diameter d, which may also be referred to as the gage or weight of the electrode. In an exemplary embodiment, a pair of electrodes 30 may be separated by a gap g, which is the distance between one electrode 30 and another across and around which an electrical field may be created.

The selection of electrode diameter g may be directly related to the gap g between electrodes. For instance, a 5 μm diameter wire will be used for an electrode configuration with 5 μm gaps g. Similarly, a 10 μm diameter electrode wire may be used in configurations with 10 μm inter-electrode spacing gaps g, and so forth. A discrete range of gap g/gages d spanning from 1 to 10 μm may be found appropriate. The voltage utilized may scale with the gap g/gages d, and may range from about 1 to 2V for the smallest gap g/gages d up to about 9V for the largest. The availability of different array and size configurations may allow the combination of materials and sizes to be tailored to specific use cases. Gap g/gages d in the range up to 6 μm may be used to attenuate micro-organismal contamination. The largest diameter gap g/gages d may be appropriate for attenuating larger-sized organisms.

A potentially suitable array 30 may be configured using interdigitated electrodes, like the ones available from MicruX Technologies, Spain (https://www.micruxfluidic.com/), and the electrodes 30 may be formed from a variety of conductive materials, including without limitation gold, silver, platinum, copper, aluminum, graphite, and conductive polymers, and combinations and alloys of conductive materials.

In an exemplary embodiment, the encapsulating material 12 may comprise a polymer. In an exemplary embodiment, the encapsulating material 12 may comprise other materials that are suitably non-conductive that may protect the electrodes 30. In an exemplary embodiment, the encapsulating material 12 may comprise other materials that may suitably hold a negative electrode 32 in relative position to a positive electrode 34, such that the pair of electrodes 30 generate an electrical field 40.

In an exemplary embodiment, the power supply 20 may include any power supply 20 that can create a charged field at levels strong enough to reduce contaminants in the immediate and surrounding surfaces, but at a level that will not impact humans. In an exemplary embodiment, the power supply 20 may create a charged field at levels strong enough to create a boundary against unwanted pests. In an exemplary embodiment, the power supply 20 may create a charged field at levels strong enough to create an efficacious zone 40.

In an exemplary embodiment, a power supply 20 may include a small photovoltaic cell that may be connected to conductive elements 30, which through alternating currents supported by a power supply 20, generate a charge harmless to human beings, yet is a significant hindrance to both viral and bacterial survival, both on the connected membrane 10 and its immediate surroundings. Even in low-level light, the material may emit enough of an electrical charge passing through its connected electrodes 30 to build an efficacious zone 40.

In an exemplary embodiment, a power source 20 may be a mechanical action from which energy may be scavenged. Repetitive motions, such as wave action or alternating cycles of compression and release, may be adapted to produce small amounts of electrical energy adequate to power a membrane 10. In some applications, a membrane in close proximity to such a mechanical action may be advantageous to the energy supply being adequate to produce an efficacious zone 40.

In an exemplary embodiment, a membrane 10 may generate an electrical field to create a hostile environment to contaminants, such as viruses and bacteria. A membrane 10 may also act as a deterrent boundary on applied surfaces, repelling non-flying insects and small animals. A material 10 may be affixed to a surface in an assortment of ways, including an adhesive, a magnet, and Van der Wall's force, which may be used on any surface with a non-zero, Gaussian curvature. Additionally, membrane 10 may be shrink-wrapped to surfaces or embedded within the surface of the design and functional components of an object.

Membrane 10 offers an efficient, effective, and economical way of keeping surfaces and environments free of viral, bacterial, and non-flying insect contamination. While the material may come in pre-formed shapes and sizes or may be integrated into the manufacturing of a product, it may also be configured to any desired shape based on consumer preference and need. This flexibility of application allows users to maintain germ-free environments wherever the membrane 10 coverage exists as well as within the reach of its resulting electromagnetic field. Given the need for effective barriers to contact contamination, the membrane 10 offers ways to mitigate the spread of pathogens.

Electric fields are known to kill toxic microorganisms such as viruses and bacteria. A study in 2008 showed that an electric field of 1 v/um was sufficient to kill bacteria (See Uchida above). Further, this study demonstrated that reducing the electrode spacing while keeping the electric field constant improved lethality against these organisms, and that pulsed operation over a wide range of pulse rates and pulse widths was effective. For example, a 200V amplitude and 100-microsecond long pulse repeated at 50 Hz across a 100-micron gap had an efficacy sufficient to kill 99% of bacteria in 20 minutes, and 99.99% of bacteria in one hour. This excitation corresponds to a 0.5% electrical duty cycle and indicates that the power consumption may be compatible with a variety of energy scavenging techniques, such as solar cells or environmental mechanical power input, such as the turning of a doorknob, turning on a light switch, swiping on a tablet, pressing an elevator call button, or stepping on a pressure switch. It is also appreciated that more unconventional power sources 20 may be suitable. It is also appreciated that other conventional power sources 20 may also be suitably employed, such as batteries and line power.

A 200V signal on bare metal conductors may not be safe for use in a public environment. Fortunately, at sufficiently small electrode 30 spacing, the voltage necessary to generate fields lethal to microorganisms becomes low enough to be safe for humans and may not be detectable during typical human contact. For example, the terminal of a 9V battery can be pressed against the skin with no concern for the state of the battery's charge because it is not noticeably detectable. A 9V potential across a five-to-ten-micron electrode gap would not be a health hazard even if the electrodes 30 were bare metal, or even if they are directly contacted to cuts, the tongue, or other sensitive skin. Moreover, the integrated use of cap charge to detect the presence of human contact may effectively, and safely, disable the electronic field.

It is also noted that very small-scale electrode 30 arrays may be printed at low cost, thanks to a variety of large-area lithography technologies developed for flat panel displays and flexible electronics. In an exemplary embodiment, arrays of electrodes 30 may be printed such that a large area is covered with many electrodes 30 wired in parallel. Interdigitated electrodes 30 are one common example. In an exemplary embodiment, electronic photovoltaic cells can be printed or otherwise fabricated using similar technology. In an exemplary embodiment, the power supply 20, the controller 18, and the electrodes 30 may all be fabricated on a single substrate.

In an exemplary embodiment, printed electrodes 30 may also be augmented with nanoparticles or nanowires. (See Liu et al., 2014, above.) Using alternating electric fields, Liu et al. showed that 99.9% of both bacteria and viruses could be killed within tens of seconds. It is also appreciated that electric fields do not require the presence of bare metal conductors. In an exemplary embodiment, conductors may be covered with a thin layer of dielectric material with very little loss in field strength.

A controller 18 may be very simple or quite complex. In some exemplary embodiments, the controller 18 may be missing entirely, and the circuitry may simply pass the voltage from the power supply 20 directly to the electrodes 30. In more complex examples, the controller 18 could control the frequency and amplitude of the voltage applied to the electrodes 30, possibly using square waves, sine waves, or other waveforms. In some embodiments, the controller 18 might use the state of the power supply 20 as an input to determine when and how to excite the electrodes 30. For example, with a weak power supply 20, the controller 18 might wait until sufficient power or energy is available from the power supply 20 before sending power to the electrodes 30. Controllers 18 might use the time of day, wireless communication via a computer application or other “smart” or “artificial intelligence” technology, or readings from sensors (such as a cap charge) to determine how and when to send power to the electrodes 30. In an exemplary embodiment, a controller 18 might contain voltage boosting, voltage regulation, or other power processing circuitry common in integrated circuits.

The innovation discloses its primary technology on a membrane 10 that may be constructed in many forms, including, without limitation, a chip, a sleeve, a sheet (customizable with various physical parameters), and a strip, each with a sterilization function. In an exemplary embodiment, a membrane 10 may be fashioned in any number of ways to provide recurring sterilization of the surfaces it covers. A user of this material, whether in the form of chip, sleeve, customizable sheet, or a strip, may be able to keep desired surfaces and environments germ and pest-free. The membrane 10 may be configured to detect the presence of human contact thus disabling the electronic field.

The innovation also discloses a coin-sized wafer, or “chip”, with the same sterilization function, which may be embedded into the structure of door handles or other metallic surfaces used for gripping. The innovation also discloses, even in low-level light, the ability to emit enough of an electrical charge passing through the connected surface at strong enough levels to create an efficacious zone 40, which may kill harmful substances, such as bacteria and viruses, and repel non-flying pests, thereby reducing threats to human health from contact with the connected surface.

Referring now primarily to FIG. 3, the membrane 10 may be employed on an assortment of high-contact items, such as door handles, latches, locks, tabletops, switches/switch-plates, touchscreen consoles, cellular phone faces and covers, as well as vehicular dashboards and other mechanical controls touched. The membrane 10 may be employed along areas to prevent wingless insects or gastropod (such as snails and slugs) infestation, or other surfaces that would benefit from being sterile, depending on preference and need.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount. Alternatively, the term “about” refers to an amount that is near the stated amount by 10%, 5%, or 1%, including increments therein. In some cases, the term “about” in reference to a percentage refers to an amount that is greater or less than the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term coating may mean a configuration of the claimed elements contained in a structure, such as a membrane 10 or other thin sheet of material, substance, or substances. The coating may be integrated into an element that forms a device. The coating may be adhered to or otherwise affixed to a device.

### Breadth of Disclosure

The foregoing disclosure and description of the invention are illustrative and explanatory thereof. The examples contained in this specification are merely possible implementations of the current device, and alternatives to the particular features and elements may be changed without departing from the spirit of the invention. The present invention should only be limited by the following claims and their legal equivalents, since the provided exemplary embodiments are only examples of how the invention may be employed and are not exhaustive.

I claim:

1. An anti-pathogen membrane system, comprising:
a power supply electrically connected to a membrane, the membrane having a negative feed line and a positive feed line;
the negative feed line electrically connected to at least one negative electrode and the positive feed line electrically connected to at least one positive electrode;
the at least one negative electrode and the at least one positive electrode arranged in an array of electrodes within an encapsulating material; and
the array of electrodes having an active mode wherein the at least one negative electrode and the at least one positive electrode are energized by the power supply to generate a charged field, the charged field creating an efficacious area extending beyond the encapsulating material.

2. The anti-pathogen membrane system of claim 1, further comprising:
a controller operatively connected the power supply.

3. The anti-pathogen membrane system of claim 2, wherein the controller controls the frequency of the power supplied to the electrodes.

4. The anti-pathogen membrane system of claim 2, wherein the controller controls the voltage of the power supplied to the electrodes.

5. The anti-pathogen membrane system of claim 2, wherein the controller activates an active mode in the electrodes in response to a timer.

6. The anti-pathogen membrane system of claim 2, wherein the controller deactivates an active mode in the electrodes in response to a timer.

7. The anti-pathogen membrane system of claim 2, wherein the controller activates an active mode in the electrodes when it senses adequate power from the power supply.

8. The anti-pathogen membrane system of claim 2, wherein the controller deactivates an active mode in the electrodes when it senses inadequate power from the power supply to maintain the efficacious area.

9. The anti-pathogen membrane system of claim 2, wherein the controller activates an active mode in the electrodes when it senses human contact with the membrane.

10. The anti-pathogen membrane system of claim 2, wherein the controller activates an active mode in the electrodes when it senses human presence in the proximity of the membrane.

11. The anti-pathogen membrane system of claim 2, wherein the controller activates an active mode in the electrodes when it senses human presence within the efficacious area.

12. The anti-pathogen membrane system of claim 2, wherein the controller is remotely controllable.

13. The anti-pathogen membrane system of claim 1, wherein the power supply being a photovoltaic cell.

14. The anti-pathogen membrane system of claim 1, wherein the power supply being a rechargeable battery.

15. The anti-pathogen membrane system of claim 1, wherein the power supply being a button battery.

16. The anti-pathogen membrane system of claim 1, wherein the power supply mechanically scavenged from the environment.

17. The anti-pathogen membrane system of claim 1, wherein the membrane being affixable to a surface.

18. The anti-pathogen membrane system of claim 1, wherein the membrane being integrated into a surface.

19. The anti-pathogen membrane system of claim 1, wherein the membrane is constructed by additive manufacturing.

20. The anti-pathogen membrane system of claim 1, wherein the membrane is constructed on a semiconductor substrate.
